(19) United States
(12) Patent Application Publication
(10) Pub. No.: US 2004/0101846 A1
(43) Pub. Date: May 27, 2004

(54) Methods for identifying suitable nucleic acid probe sequences for use in nucleic acid arrays

(76) Inventors: Patrick J. Collins, Daly City, CA (US); Anna M. Tsalenko, Chicago, IL (US); Zohar H. Yakhini, Ramat Hasharon (IL); Peter G. Webb, Menlo Park, CA (US); Karen W. Shannon, Los Gatos, CA (US); Stephanie B. Fulmer-Smentek, Sunnyvale, CA (US)

Correspondence Address: AGILENT TECHNOLOGIES, INC., INTELLECTUAL PROPERTY ADMINISTRATION, LEGAL DEPT., P.O. BOX 7599, M/S DL429, LOVELAND, CO, 80537-0599, US

(21) Appl. No.: 10/303,160

(22) Filed: Nov. 22, 2002

(51) Int. Cl. C12Q 1/68; G06F 19/00; G01N 33/48; G01N 33/50

(52) U.S. Cl. 435/6; 702/20

(57) ABSTRACT

Methods of identifying a sequence of a probe, e.g., a biopolymeric probe, such as a nucleic acid, that is suitable for use as a surface immobilized probe for a target molecule of interest, e.g., a target nucleic acid, are provided. A feature of the subject methods is that a set of computationally determined initial candidate sequences are empirically evaluated to obtain functional data that is then employed to identify one or more clusters of candidate probe sequences from the initial set such that all candidate probe sequences within each identified cluster exhibitsubstantially the same performance under a plurality of different experiments, specifically a plurality of differential gene expression experiments. A candidate probe from the cluster that exhibits the best performance across the plurality of experimental sets is then selected as the optimum candidate probe, e.g., based on one or more performance metrics. The subject invention also includes algorithms for performing the subject methods recorded on a computer readable medium, as well as computational analysis systems that include the same. Also provided are nucleic acid arrays produced with probes having sequences identified by the subject methods, as well as methods for using the same.

### FIELD OF THE INVENTION

The field of this invention is nucleic acid arrays.

### BACKGROUND OF THE INVENTION

Arrays of binding agents or probes, such as polypeptide and nucleic acids, have become an increasingly important tool in the biotechnology industry and related fields. These binding agent arrays, in which a plurality of probes are positioned on a solid support surface in the form of an array or pattern, find use in a variety of different fields, e.g., genomics (in sequencing by hybridization, SNP detection, differential gene expression analysis, identification of novel genes, gene mapping, finger printing, etc.) and proteomics.

In using such arrays, the surface bound probes are contacted with molecules or analytes of interest, i.e., targets, in a sample. Targets in the sample bind to the complementary probes on the substrate to form a binding complex. The pattern of binding of the targets to the probe features or spots on the substrate produces a pattern on the surface of the substrate and provides desired information about the sample. In most instances, the targets are labeled with a detectable label or reporter such as a fluorescent label, chemiluminescent label or radioactive label. The resultant binding interaction or complexes of binding pairs are then detected and read or interrogated, for example by optical means, although other methods may also be used depending on the detectable label employed. For example, laser light may be used to excite fluorescent labels bound to a target, generating a signal only in those spots on the substrate that have a target, and thus a fluorescent label, bound to a probe molecule. This pattern may then be digitally scanned for computer analysis.

Generally, in discovering or designing probes to be used in an array, a nucleic acid sequence is selected based on the particular gene of interest, where the nucleic acid sequence may be as great as about 60 or more nucleotides in length or as small as about 25 nucleotides in length or less. From the nucleic acid sequence, probes are synthesized according to various nucleic acid sequence regions, i.e., subsequences, of the nucleic acid sequence and are associated with a substrate to produce a nucleic acid array. As described above, a detectably labeled sample is contacted with the array, where targets in the sample bind to complimentary probe sequences of the array.

As is apparent, a key step in designing arrays is the selection of a specific probe or mixture of probes that may be used in the array and which maximize the chances of binding with a specific target in a sample, while at the same time minimize the time and expense involved in probe discovery and design. In practice, designing an optimized array typically involves iterating the array design one or more times to replace probes that are found to be undesirable for detecting targets of interest, either due to poor signal quality and/or cross-hybridization with sequences other than the targets of interest. Such iterations are costly and time consuming.

For example, conventional probe design may be performed experimentally or computationally, where in many instances,it is performed computationally Accordingly, probe design usually involve staking subsequences of a nucleic acid and filtering them based on certain computationally determined values such as melting temperature, self structure, homology, etc., to attempt to predict which subsequences will generate probes that will provide good signal and/or will not cross-hybridize. The subsequences that remain after the filtering process are selected to generate probes to be used in nucleic acid arrays.

While attempts have been made to predict which probes will provide the best results in an array assay, such attempts are not completely satisfactory as probes selected using these methods are often still found to be undesirable for one or both of the above-described reasons. In other words, some probes will still fail or give false results as the computational techniques used to filter and select the probes are not precise predictors. Accordingly, as mentioned above, typically an array deign must be iterated a number of times in order to filter out all the undesirable probes from the array. Furthermore, such attempts often characterize probes after they have been synthesized, that is after time and expense have been already been invested.

There is continued interest in the development of new methods and devices for producing arrays of nucleic acid probes that provide strong signal and do not cross hybridize with sequences other than targets of interest.

Relevant Literature

U.S. Patents of interest include: U.S. Pat. Nos. 6,251,588 and 5,556,749. Also of interest is Hosaka et al., Genome Informatics (2001) 12: 449-450.

### SUMMARY OF THE INVENTION

Methods of identifying a sequence of a probe, e.g., a biopolymeric probe, such as a nucleic acid, that is suitable for use as a surface immobilized probe for a target molecule of interest, e.g., a target nucleic acid, are provided. A feature of the subject methods is that a set of computationally determined initial candidate sequences are empirically evaluated to obtain functional data that is then employed to identify one or more clusters of candidate probe sequences from the initial set such that the selected cluster members exhibit substantially the same performance across a plurality of different experimental sets, specifically a plurality of differential gene expression experiments. The “best” cluster of candidate probes is determined based on at least one criterion, such as cluster size or cluster score, or a combination thereof. One optimum candidate probe sequence is then selected from the “best” cluster of candidate probes on the basis of empirically measured performance metrics. The subject invention also includes algorithms for performing the subject methods recorded on a computer readable medium, as well as computational analysis systems that include the same. Also provided are nucleic acid arrays produced with probes having sequences identified by the subject methods, as well as methods for using the same.

### BRIEF DESCRIPTIONS OF THE DRAWING

FIG. 1 shows a flowchart representing the steps of the subject methods.

### DEFINITIONS

In the present application, unless a contrary intention appears, the following terms refer to the indicated characteristics.

The term “polymer” means any compound that is made up of two or more monomeric units covalently bonded to each other, where the monomeric units may be the same or different, such that the polymer may be a homopolymer or a heteropolymer. Representative polymers include peptides, polysaccharides, nucleic acids and the like, where the polymers may be naturally occurring or synthetic.

The term “biopolymer” refers to a polymer of one or more types of repeating units. Biopolymers are typically found in biological systems and particularly include polysaccharides (such as carbohydrates), and peptides (which term is used to include polypeptides and proteins) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or,synthetic) capable of participating in Watson-Crick type hydrogen bonding interactions. Polynucleotides include single or multiple stranded configurations, where one or more of the strands may or may not be completely aligned with another. For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source.

The term “nucleic acid” as used herein means a polymer composed of nucleotides, e.g., deoxyribonucleotides or ribonucleotides, or compounds produced synthetically (e.g. PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” refers to a nucleotide multimer of about 10 to 100 nucleotides in length and up to 200 nucleotides in length.

The term “polynucleotide” as used herein refers to a nucleotide multimer having any number of nucleotides.

The term “biomonomer” references a single unit, which can be linked with the same or other biomonomers to form a biopolymer (for example, a single amino acid or nucleotide with two linking groups one or both of which may have removable protecting groups). The terms “biomonomer fluid” and “biopolymer fluid” reference a liquid containing either a biomonomer or biopolymer, respectively (typically in solution).

The term “monomer” as used herein refers to a chemical entity that can be covalently linked to one or more other such entities to form a polymer. Examples of “monomers” include nucleotides, amino acids, saccharides, peptides, other reactive organic molecules and the like. In general, the monomers used in conjunction with the present invention have first and second sites (e.g., C-termini and N-termini(for proteins), or 5 ′ and 3′ sites(for oligomners, RNA's, cDNA's, and DNA's)) suitable for binding to other like monomers by means of standard chemical reactions (e.g., condensation, nucleophilic displacement of a leaving group, or the like), and a diverse element which distinguishes a particular monomer from a different monomer of the same type (e.g., an amino acid side chain, a nucleotide base, etc.). In the art synthesis of biomolecules of this type utilize an initial substrate-bound monomer that is generally used as a building-block in a multi-step synthesis procedure to form a complete ligand, such as in the synthesis of oligonucleotides, oligopeptides, and the like.

The term “oligomer” is used herein to indicate a chemical entity that contains a plurality of monomers. As used herein, the terms “oligomer” and “polymer” are used interchangeably. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure.

The term “sample” as used herein relates to a material or mixture of materials, typically, although not necessarily, in fluid form, containing one or more targets, i.e., components or analytes of interest.

The terms “nucleoside” and “nucleotide” refer to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. The terms “nucleoside” and “nucleotide” are intended to include those moieties which contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms, “may” “optional” or “optionally” used herein interchangeably means that the subsequently described circumstance may or may not occur, so that the description includes instances where the circumstance occurs and instances where it does not.

The terms “probe”, “probe sequence”, “target probe” or “ligand” as used herein refer to a moiety made of an oligonucleotide or polynucleotide, as defined above, which contains a nucleic acid sequence complementary to a nucleic acid sequence present in a sample of interest such that the probe will specifically hybridize to the nucleic acid sequence present in the sample under appropriate conditions. The nucleic acid probes of the subject invention are typically associated with a support or substrate to provide an array of nucleic acid probes to be used in an array assay. The term “probe” or its equivalents as used herein refer to a compound that is “pre-synthesized” or obtained commercially, and then attached to the substrate or synthesized on the substrate, i.e., synthesized in situ on the substrate. The nucleic acid probes of the subject invention are produced, generated or synthesized according to probe sequences identified as suitable according to the subject invention that may or may not have been further tested or characterized.

The terms “reporter”, “label” “detectable reporter” and “detectable label” are used herein to refer to a molecule capable of detection, including, but not limited to, radioactive isotopes, fluorescers, chemiluminescers, enzymes, enzyme substrates, enzyme cofactors, enzyme inhibitors, dyes, metal ions, metal sols, other suitable detectable markers such as biotin or haptens and the like. The term “fluorescer” refers to a substance or portion thereof which is capable of exhibiting fluorescence in the detectable range. The term “cofactor” is used broadly herein to include any molecular moiety that participates in an enzymatic reaction. Particular example of labels which may be used under the invention include, but are not limited to, fluorescein, 5(6)-carboxyfluorescein, Cyanine 3 (Cy3), Cyanine 5 (Cy5), rhodamine, dansyl, umbelliferone, Texas red, luminal, NADPH, horseradish peroxidase and α, β-galactosidase.

An “array,” includes any two-dimensional or substantially two-dimensional (as well as a three-dimensional) arrangement of addressable regions bearing a particular chemical moiety or moieties (e.g., biopolymers such as polynucleotide or oligonucleotide sequences (nucleic acids), polypeptides (e.g., proteins), carbohydrates, lipids, etc.) associated with that region. In the broadest sense, the preferred arrays are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. Nos. 6,242,266, 6,232,072, 6,180,351, 6,171,797, 6,323,043, U.S. patent application Ser. No. 09/302,898 filed Apr. 30, 1999 by Caren et al., and the references cited therein. These references are incorporated herein by reference. Other drop deposition methods can be used for fabrication, as previously described herein. Also, instead of drop deposition methods, photolithographic array fabrication methods may be used such as described in U.S. Pat. Nos. 5,599,695, 5,753,788, and 6,329,143. Interfeature areas need not be present particularly when the arrays are made by photolithographic methods as described in those patents.

An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions However, either of the “target” or “target probe” may be the one which is to be evaluated by the other (thus, either one could be an unknown mixture of polynucleotides to be evaluated by binding with the other). A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas that lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “stringent hybridization conditions” as used herein refers to conditions that are that are compatible to produce duplexes on an array surface between complementary binding members, i.e., between probes and complementary targets in a sample, e.g., duplexes of nucleic acid probes, such as DNA probes, and their corresponding nucleic acid targets that are present in the sample, e.g., their corresponding mRNA analytes present in the sample. An example of stringent hybridization conditions is hybridization at 60° C. or higher and 3×SSC (450 mM sodium chloride/45 mM sodium citrate). Another example of stringent hybridization conditions is incubation at 42° C. in a solution containing 30% formamide, 1M NaCl, 0.5% sodium sarcosine, 50 mM MES, pH 6.5. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

### DETAILED DESCRIPTION OF THE INVENTION

Methods of identifying a sequence of a probe, e.g., a biopolymeric probe, such as a nucleic acid, that is suitable for use as a surface immobilized probe for a target molecule of interest, e.g., a target nucleic acid, are provided. A feature of the subject methods is that a set of computationally determined initial candidate sequences are empirically evaluated to obtain functional data that is then employed to identify one or more clusters of candidate probe sequences from the initial set such that all candidate probe sequences within each identified cluster exhibit substantially the same performance under a plurality of different experiments, specifically a plurality of differential gene expression experiments. A candidate probe from the cluster that exhibits the best performance across the plurality of experimental sets is then selected as the optimum candidate probe, e.g., based on one or more performance metrics. The subject invention also includes algorithms for performing the subject methods recorded on a computer readable medium, as well as computational analysis systems that include the same. Also provided are nucleic acid arrays produced with probes having sequences identified by the subject methods, as well as methods for using the same.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting Instead, the scope of the present invention will be established by the appended claims.

In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described. Methods recited herein may be carried out in any order of the recited events which is logically possible, as well as the recited order of events.

All patents and other references cited in this application, are incorporated into this application by reference except insofar as they may conflict with those of the present application (in which case the present application prevails).

As summarized above, the subject invention provides methods of identifying or designing probes for use in an array structures, where the probes are chemical probes, e.g., biopolymeric probes, such as nucleic acids. While the following description is provided in terms of nucleic acid probe design protocols for ease and clarity of description, the scope of the invention is not so limited, but instead extends to the identification or design of suitable probes for use in any type of array structure.

In further describing the subject invention, the methods of identifying suitable probe sequences are described first in greater detail, followed by a review of arrays that may be produced using probes identified by the subject methods as well as representative applications for such arrays.

Methods

As summarized above, the subject invention provides methods of identifying a sequence of a nucleic acid that is suitable for use as a surface immobilized probe for a target nucleic acid. In other words, the subject invention provides methods of designing nucleic acid probes that are suitable for use on nucleic acid arrays. The subject methods result in the identification of a set or cluster of probes, where the set or cluster of probes identified using the subject methods are suitable for use as array probes because the selected cluster members exhibit substantially the same performance across a plurality of different experimental sets, specifically a plurality of differential gene expression experiments. A feature of the subject methods is that they include both computational steps and empirical steps, where specifically a collection of candidate probe sequences for a given target nucleic acid are first computationally identified from the sequence of the target nucleic acid of interest, where the initially identified candidate sequences are subsequently tested empirically and then further evaluated using additional computational steps in order to identify a suitable set or collection of probe sequences from which “best” probe sequences may be selected..

In many embodiments, the subject methods include the following steps:

(a) identifying a plurality of candidate probe sequences for the target nucleic acid;

(b) empirically evaluating each of the identified candidate probe sequences;

(c) clustering the identified candidate probe sequences into two or more groups of candidate probe sequences based observed empirical data values, where clustered members exhibit substantially the same performance across a plurality of different experimental sets, specifically a plurality of differential gene expression experiments;

(d) selecting one of the two or more groups of candidate probe sequences as the “best” group; and

(e) choosing a candidate probe sequence from the selected “best” group as the sequence that is most suitable for use as a probe for the target nucleic acid of interest.

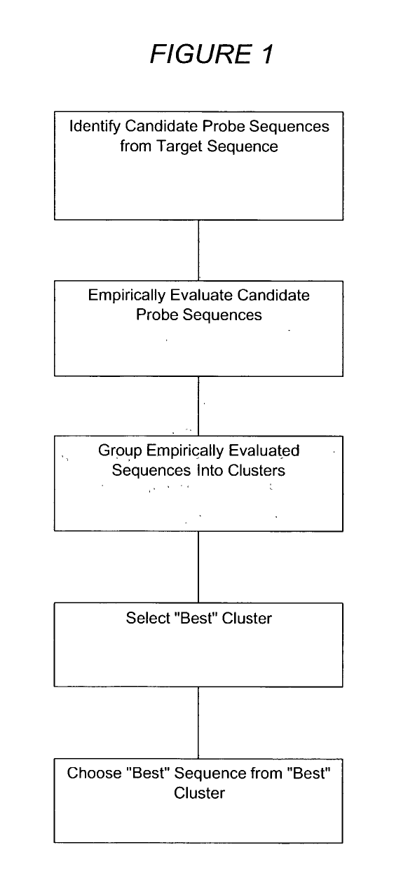

FIG. 1 provides a flow chart showing each of the above steps of the subject methods. In further describing the subject methods, each of the above steps is now reviewed separately in greater detail below.

Candidate Probe Identification

As mentioned above, the first step in the subject methods is to identify a plurality of candidate probe sequences for a given target nucleic acid of interest. The target nucleic acid of interest is generally a nucleic acid of known sequence, where the length of the nucleic acid may vary, but typically ranges from about 200 nt to about 4,000 nt, such as from about 400 nt to about 2,500 nt, including from about 800 nt to about 1,500 nt. In many embodiments, the target nucleic acid has the sequence of an mRNA transcript of interest or the complementary sequence thereof, or the sequence of a first or second strand DNA prepared from an mRNA of interest.

The candidate probes are identified based on a least one selection criterion, wherein in many embodiments a plurality of different selection criteria are employed together to identify the candidate probes from the target nucleic acid sequence, where by plurality is meant at least about 2, and may be as greater as 10 or more, but is typically less than 5, e.g., 2 to 3.

One selection criterion of interest that may be employed is distance from the 3′-end of the mRNA transcript that corresponds to the target nucleic acid, e.g., that is the target nucleic acid or is the complement of the target nucleic acid, or from which the target nucleic acid is derived, e.g., where the target nucleic acid is first or second strand cDNA. When this criterion is employed, candidate sequences of the target nucleic acid are chosen that are within at least about 2,000 nt, usually within about 1,500 nt and more usually within about 800 nt of the 3′ end of the mRNA that corresponds to the target nucleic acid..

Another selection criterion of interest is the base composition of the probe sequence. When this criterion is employed, sequences that are abnormally GC rich or poor, long runs of a single base, and/or base compositions that are known to generate unacceptable array features, e.g., under in situ production conditions are avoided. Sequences that are abnormally GC rich or poor are those sequences whose number % of G and C bases are greater than about 30, such as greater than about 35, or less than about 60, such as less than about 45. By “long run” of a single base is meant a stretch of nucleotides of the same base that is greater than about 6, such as greater than about 10. Sequences that are known to generate unacceptable array features include, but are not limited to those containing stretches of at least 10 Gs.

Another selection criterion of interest is homology of the candidate probe sequence to other sequences from the same organism, i.e., to other mRNA transcripts or complements thereof of the same organism from which the target sequence of interest for which the probe is being designed is obtained. Sequences with a high potential to hybridize to more than one mRNA transcript from a given organism are avoided. Cross-hybridization potential of candidate sequences may be estimated via thermodynamic scoring of the output of BLAST, a standard bioinformatics application used to detect sequence homology and well known to those of skill in the art, or any other convenient cross-hybridization potential assessment protocol. Use of this criterion results in the identification of probe sequences that are specific for the target nucleic acid of interest.

In certain embodiments, the identification process or algorithm that is employed is one in which parameters are used that minimize the number of identified candidate probe sequences that overlap with each other. Any of the above listed criteria may be adjusted in order to result in minimal overlap of the identified candidate probe sequences. The overlap parameter is designed to yield candidate probes that span the target—if it is not specified, the algorithm employed, may identify probes that are heavily overlapped (up to 59 out of 60 bases). While these may be the best probes, using such a set of candidates confounds the clustering analysis, since almost by definition such probes will cluster tightly.

Using the above protocol, a plurality of candidate probe sequences are identified for a given target nucleic,acid. In many embodiments, the number of identified candidate probe nucleic acid sequences is at least about 5, usually at least about 7 and may be as great as 15, 20 or more, but typically does not exceed about 15, where in certain embodiments, the number of candidate probe sequences identified for a given target nucleic acid ranges from about 7 to 12, e.g., 8,9,10 or 11.

In certain embodiments, an algorithm is employed, e.g., in conjunction with a computational analysis system, to identify candidate probe sequences from a target nucleic acid. Any convenient algorithm or process capable of performing the above function may be employed. Of interest in many embodiments are the Agilent probe design algorithms (Agilent Technologies, Palo Alto, Calif.), where the algorithms are employed in identification of candidate probe sequences. Specifically, the design parameters that may be employed include: 1) the preferred and allowed distances from the 3′ end, 2) the number of probes required before ending base composition iteration (where a suitable number typically ranges from about 20 to about 200, usually from about 50 to about 100 ), 3) the criteria used to label probes as “overlap” (where “overlap” may be defined as probes whose sequences overlap by a number of bases, for example greater than 10 nt, more typically greater than 40 nt), and 4) the number of probes required before the homology calculation (where a suitable number typically ranges from about 10 to about 40, usually from about 12 to about 20).

As indicated above, the above first step in the subject methods results in the identification of a plurality of different candidate probe sequences for a given target nucleic acid.

Empirical Evaluation of Identified Candidate Probe Nucleic Acid Sequences

In the next step of the subject methods, each of the identified candidate probe sequences are evaluated empirically. Specifically, each of the identified candidate probe sequences are evaluated for their performance under a plurality of different experimental sets, specifically a plurality of differential gene expression experiments to obtain a collection of empirically obtained performance data values for each of the candidate nucleic acid probe sequences for each of the plurality of different experimental conditions. In many embodiments, the experimental conditions are differential gene expression assay experiments, where a given experimental condition is a differential gene expression assay using a particular nucleic acid sample pair, where each sample of the pair is obtained from a different source, e.g., tissue or cell line. Differential gene expression array based assays are well known to those of skill in the art. The number of different differential gene expression array based assays for which a given candidate probe is empirically evaluated may vary, where the number may range from about 2 to about 20, such as from about 5 to about 15, including from about 7 to about 12, e.g., 10. Any two differential gene expression assays or protocols are considered different if at least one of the nucleic acid samples making up the pairs of any two pairs differs between the two pairs.

The differential gene expression assays are typically performed by first providing an array of candidate nucleic acid probes immobilized on a surface of a solid support, where the array includes a substrate surface immobilized nucleic acid candidate probe for each of the identified candidate probe sequences to be empirically evaluated. In other words, an array is provided that includes a probe for each of the to be evaluated candidate probe sequences, i.e., all of the to be evaluated candidate probe sequences have corresponding probes on the array that include the same sequence. The arrays of candidate probes may be provided in a number of different ways, e.g., via in situ production, as described in U.S. Pat. Nos. 6,451,998; 6,446,682; 6,440,669; 6,420,180; 6,372,483; 6,323,043; and 6,242,266; the disclosures of which patents are herein incorporated by reference.

The surface immobilized candidate probes having the sequences of the candidate probe sequences are then contacted with two or more sets of nucleic acid sample pairs under differential gene expression analysis conditions to evaluate the probes. In certain embodiments, an identical candidate probe array is contacted with each different sample pair of the set of different sample pairs, while in other embodiments, the same nucleic acid array may be contacted with two or more sample pairs, so long as any hybridized targets from any previous assay are efficiently removed or “stripped” prior to contact with the next sample pair. Differential gene expression assay protocols are further described below.

In a representative example of the above empirical evaluation step of the subject methods, multiple copies of a microarray that includes candidate 60-mer probes having sequences identified by the prior sequence identification step are produced using an in situ nucleic acid array synthesis protocol. These resultant microarrays are then hybridized to 10 different tissue/cell line combinations (4 replicates per sample pair): one self-vs.-self and 9 sample pairs chosen to maximize the number of mRNAs that are differentially expressed between the members of the pair. The arrays are then scanned, as described in greater detail below, and the feature data are extracted using extraction software, such as Agilent's Feature Extraction software (available from Agilent Technologies, Palo Alto, Calif.). Where desired, the resultant data may be placed in tabular form or collated into a relational database or otherwise organized. Typically, the feature extraction protocol computes P-values, specifically the likelihood that the P-value is significantly different from 0. The feature data are further processed to exclude data from features that do not satisfy certain quality control measures, e.g., signal saturation or the presence of too many outlier pixel values and to exclude data from probes that do not generate sufficient signal in any of the experiments. The obtained feature data are further processed by combining replicate experiments using statistical weights derived from the P-values associated with each feature, e.g., by using a processing algorithm designed for this purpose.

The above empirical evaluation process results in the production of a collection of empirically obtained data values for each candidate probe sequence, where the empirical data values are measures of performance across a plurality of different experimental sets, specifically a plurality of differential gene expression experiments. Specifically, a collection of probe performance data values (e.g., in the form of log ratio values) for each different differential gene expression experiment is obtained for each candidate probe, such that for each probe one obtains an empirical or experimentally determined measure of that probe's performance in each of a number of different differential gene expression assays, e.g., a value is obtained to represent performance of each probe in each experiment. The data making up a given collection of data values may be raw data or processed, and may be a measure of hybridization efficiency, signal intensity, signal ratio, signal log-ratio or combination thereof.

Clustering Candidate Probe Sequences

In the next step of the subject methods, the candidate probe sequences are clustered into two or more groups of candidate probe sequences, where the candidate probe sequences are divided into two or more groups of candidate probe sequences based on the observed empirical data values obtained in the prior empirical evaluation step.

In many embodiments of this clustering or grouping step, one first obtains an expression vector for each of the candidate probe sequences using the candidate probe sequence's collection of empirical data values. From the obtained expression vector for each candidate probe sequence, one then derives a similarity matrix for the set of the candidate probe sequences, where the similarity matrix provides a measure of how similar the candidate probe sequence functions as compared to the other candidate probe sequences being evaluated. Based on the derived similarity matrix for the set of candidate probe sequences, the candidate probe sequences are then grouped into two or more groups. Each of the above substeps of the clustering step is now reviewed separately in greater detail.

As indicated above, the first substep of the clustering step is the generation of an expression vector for each candidate probe sequence, where the expression vector is generated using the empirical data for the candidate probe sequence obtained in the empirical evaluation step described above. In many embodiments, the empirical data employed in the generation of the expression vector are the log ratio values from the sample-pair experiments, as indicated above. Where present, replicate log ratio values may be combined using error-weighted averaging. The combined log ratio data for candidate probes designed to target a single gene are used to populate an expression matrix I, where Iᵢⱼ is the measured expression level of probe i in experiment (condition) j. The number of columns in the expression matrix is the number of experiments performed for empirical validation, the number of rows in the expression matrix is the number of candidate probes designed to target a single gene. The significance of the similarity measure used depends on the number of experimental conditions performed. When Pearson correlation is used to measure the similarity of probes, the expression matrix should consist of at least 4 experiments, preferably 8 experiments, and even more preferably of at least 12 experiments. The matrix contains only data that survive the processing steps described above. As indicated above, certain feature data may be excluded, leading to missing values in the expression matrix, typically indicated by entering a special value (one that could never arise from an experiment, for example a log ratio of 10\hat{ }6) into the matrix. Subsequent processing steps must be able to process such a matrix.

In the next substep, a similarity matrix is derived or calculated from the obtained expression matrix of the first substep. In this similarity matrix, the entry Sᵢⱼ represents the similarity of the expression vectors for probes i and j. The similarity measure used for this step is independent of the clustering mechanism. Specific examples are Pearson's correlation coefficient (as described in Duda, R. O., and Hart, P. E. (1973). Pattern Classification and Scene Analysis. New York, John Wiley and Sons.), Kendall's rank correlation (as described in Kendall, M. G. (1970). Rank Correlation Methods (4^{th} edition). Griffin and Co. Ltd.), similarity measure based on the Euclidian distance, and weighted Pearson 's correlation.

Specific details on the above are provided below:

Let P be the expression matrix with m rows and n columns. Entry Pᵢⱼ of this matrix is the expression level of probe i in the experiment j. The entry Sᵢⱼ of similarity matrix S is the similarity between probe i and probe j, specific examples of how the similarity may be computed are given below.

1. Pearson's correlation.

Duda, R. O., and Hart, P. E. (1973). Pattern Classification and Scene Analysis. New York, John Wiley and Sons.

Pearson's correlation Sᵢⱼ between probes i and j is

$$\begin{matrix}{S}_{ij} = \frac{{\sum }_{k = 1}^{n} ( {P}_{ik} - {P}_{i} ) ( {P}_{jk} - {P}_{j} )}{{σ}_{i} {σ}_{j}} , where \\ {P}_{i} = \frac{1}{n} {\sum }_{k = 1}^{n} {P}_{ik} , \\ {σ}_{i}^{2} = \frac{1}{n} {\sum }_{k = 1}^{n} {( {P}_{ik} - {P}_{i} )}^{2} .\end{matrix}$$

2. Kendall's rank correlation.

Kendall, M. G. (1970). Rank Correlation Methods (4ₜₕ edition). Griffin and Co. Ltd.

3. Euclidean distance converted to similarity measure.

$${E}_{ij} = \sqrt{{\sum }_{k = 1}^{n} {( {P}_{ik} - {P}_{jk} )}^{2}}$$

$$Let min E = {min}_{{1 \leq i \leq m}_{1 \leq j \leq n}} {E}_{ij} and max E = {max}_{{1 \leq i \leq m}_{1 \leq j \leq n}} {E}_{ij} . \\ Then$$

$${S}_{ij} = 1 - \frac{{E}_{ij} - min E}{max E - min E} .$$

4. Weighted Pearson's correlation.

Analogous to Pearson's correlation, but each experiment j is taken with weight wⱼ.

Given n weights w₁, w₂, . . . , wₙ , such that Σⱼ₌₁ⁿwⱼ=1, weighted Pearson's correlation is computed in the following way:

$${S}_{ij} = \frac{{\sum }_{k = 1}^{n} ( {w}_{k} {P}_{ik} - {P}_{i} ) ( {w}_{k} {P}_{jk} - {P}_{j} )}{{σ}_{i} {σ}_{j}} ,$$

where Pᵢ and σᵢ are weighted mean and standard deviation of the probe i:

$$\begin{matrix}{P}_{i} = \frac{1}{n} {\sum }_{k = 1}^{n} {w}_{k} {P}_{ik} . \\ {σ}_{i}^{2} = \frac{1}{n} {\sum }_{k = 1}^{n} {( {w}_{k} {P}_{ik} - {P}_{i} )}^{2} .\end{matrix}$$

In the third substep, the candidate probes are clustered into one or more groups based on their similarity indices or matrices, as determined in the previous substep. In other words, the candidate probe sequences are placed into groups based on similar expression patterns. In this substep, a clustering algorithm is typically employed. Several clustering approaches can be applied here, where certain embodiments use the following approach. The input to the algorithm is a pair (S,t) where S is a n-by-n similarity matrix (n is equal to the number of candidate probes and ranges from about 3 to about 20, usually from about 5 to about 12) and t is a user-specified affinity threshold that determines what affinity level is considered significant (where t often ranges from about 0.3 to about 0.9, such as from about 0.5 to about 0.8). The algorithm constructs clusters incrementally and uses average inter-cluster similarity (affinity) between unassigned vertices and the current cluster to make its next decision to add or remove elements from groups. The clusters are “stable” when the average similarity exceeds the affinity threshold (t). In many embodiments, the algorithm allows input of up to 5 t values and iteratively performs the cluster analysis at decreasing affinity thresholds until a cluster of a user-defined minimum size is formed. Cluster members are assigned for each cluster and a cluster size and a cluster quality score is calculated. The quality score of a cluster is a measure of the likelihood of such a cluster occurring if data from unrelated probes from the data set were clustered. Highly unlikely clusters (i.e., those where the data cluster much more tightly than would be expected from data randomly selected according to the distribution of similarity between all probes in the data) are given high scores.

The above clustering protocol and substeps thereof, including the specific representative clustering protocol above that includes affinity value and scoring features) may be performed using any convenient algorithm. Of interest are algorithms that automate the steps of data filtering, data combination, clustering, cluster filtering, and probe selection, e.g., by performing all of the above described substeps. Of particular interest are algorithms that form a non-hierarchical clustering (i.e., the clusters are unrelated and cluster boundaries are determined by the algorithm) and do not assume a given number of clusters (i.e., the number of clusters is determined by the algorithm instead of being a constant given as an input parameter). In certain embodiments, the algorithm employed in this step is a CAST (Cluster Affinity Search Technique) clustering algorithm, as known to those of skill in the art and described in U.S. Pat. No. 6,421,668; the disclosure of which is herein incorporated by reference. See also U.S. Pat. No. 6,468,476, the disclosure of which is herein incorporated by reference, which further discloses Clustering programs or algorithms that may find use in the subject methods.

The above substep results in clustering or grouping of the different candidate probe sequences into two or more groups or clusters of sequences, where each cluster is made up of probe sequences that hybridize to a single target and behave similarly in gene expression experiments, both within a single experimental sample pair and across multiple sample experimental pairs.

Selection of the “Best” Cluster”

In the next step of the subject methods, a cluster or group produced in the preceding step is identified or selected, i.e., chosen, as the “best” group or cluster, based on at least one criterion, such as affinity threshold, cluster size, cluster score, or combination thereof.

In many embodiments, the protocol employed in this step identifies the “best cluster” based on the affinity threshold used and the size or score of the cluster formed. There are two underlying assumptions in this selection: 1) the expression patterns for the candidate probes are representative of the expression patterns for all the possible probes for a given target; and 2) candidate probes that show gene expression pattern(s) that differ from the pattern shown by the majority of the candidate probes are “outliers”. Thus, for those targets where the candidate probe sequences partition into multiple groups, the goal of the selection strategy of this step is to identify clusters that include a majority of the candidate probes tested. Two representative selection schemes that achieve this goal are described in the examples below. In the first scheme, the “representative (best) cluster” is chosen as the cluster formed at the highest t value that allowed formation of a cluster with at least 5 elements, where elements means candidate probes. These criteria are chosen so that “representative clusters” include at least 60% of the probe sequences tested for a given target sequence, and is twice as large as the second largest cluster. In the second scheme, the “representative cluster” is made up of at least 50% of the candidate probe sequences.

Selection of Optimum Candidate Probe Sequence

In the final step of the subject methods, a probe sequence that can be employed in a probe suitable for use as a surface immobilized probe for the target of interest is selected from the identified “best” cluster in the previous step. In other words, a “best” probe sequence is selected from previously identified “best” cluster of candidate probe sequences. (For those targets where a representative cluster is not identified, an optimal probe may be chosen using other criteria, e.g., using the computationally predicted performance of the probes.

In many embodiments, the protocol employed in this step identifies a probe sequence from the representative cluster based on empirical data that demonstrate that the probe is meeting a minimum performance metric. Such metrics may include signal intensities (or processed signal intensities), the confidence measures of the gene expression values, or some combination thereof. Two representative selection schemes are described in greater detail below.

In certain embodiments, a series of filters are applied sequentially. For example, the formation of clusters and selection of a best cluster may be viewed as the first filter. The next filter may be, ranked p-value. Then, the final filter may be based on signal. At each step, probes may be removed that to not meet minimum values, but those that do are not distinguished from each other. In the filtering by P-value step, for example, the binning will specify that any probe with certain performance characteristics is acceptable, and not rank those that are found acceptable. Only on the very last selection step, in this case signal level, is a selection of one probe made.

In the first selection scheme, the probe sequence is selected from the previously identified representative cluster using the confidence measures of the gene expression values for each probe across the experimental set, as computed by an appropriate algorithm, such as the Agilent Feature Extraction program (available from Agilent Technologies, Palo Alto, Calif.), and the maximum signal intensity value obtained for each probe sequence. The initial selection criteria selects candidate probes showing the largest number of gene expression values of highest confidence across the experimental set. P-values are calculated for each combined log ratio value as the probability that the probe shows no differential expression. The P-values are binned by a user-defined P-value threshold so that all P-values within the threshold are equivalent. For each tissue/cell line combination, the candidate probes for each target are ranked by the binned P-values. For each candidate probe, the sum of the ranks is calculated and the candidate probes showing equivalent “sum of ranked P-values” become candidate “OptProbes” that pass onto the next selection criteria. The OptProbe (i.e., the “best” probe) is selected from the candidate OptProbes based on the signal intensity; the maximum mean signal intensity is calculated for each probe across the experimental set and the optimal probe is selected from the candidates as the probe showing the highest maximum mean signal intensity.

In the second scheme, the OptProbe is selected from the representative cluster by choosing the probe showing the minimum (or average or median) P-value across the experimental set.

The above described methodology results in the selection of probe sequences for use in surface immobilized probes that show minimal, if any cross-hybridization.

In many embodiments, the probe nucleic acid sequences identified using the subject methods are provided in text format or as a string of text, where the text represents or corresponds to the sequence of nucleotides of a probe nucleic acid. The nucleic acid sequences can be of any length, where the nucleic acid sequences are typically about 20 nt to about 100 nt in length, e.g., from about 20 to about 80 nt in length, e.g., 25 nt, 60 nt, etc. However, nucleic acid sequences of lesser or greater length may be identified as appropriate. Suitable nucleic acid probes produced therefrom may be oligonucleotides or polynucleotides, as will be described in greater detail below.

One or more aspects of the above methodology may be in the form of computer readable media having programming stored thereon for implementing the subject methods. The computer readable media may be, for example, in the form of a computer disk or CD, a floppy disc, a magnetic “hard card”, a server, or any other computer readable media capable of containing data or the like, stored electronically, magnetically, optically or by other means. Accordingly, stored programming embodying steps for carrying-out the subject methods may be transferred to a computer such as a personal computer (PC), (i.e., accessible by a researcher or the like), by physical transfer of a CD, floppy disk, or like medium, or maybe transferred using a computer network, server, or other interface connection, e.g., the Internet.

In one embodiment of the subject invention, a system of the invention may include a single computer or the like with a stored algorithm capable of carrying out suitable probe identification methods, i.e., a computational analysis system. In certain embodiments, the system is further characterized in that it provides a user interface, where the user interface presents to a user the option of selecting among one or more different, including multiple different, inputs, e.g., various parameter values for the algorithm, as described above, such as distance from 3′ end, definition of overlap, t, etc. Computational systems that may be readily modified to become systems of the subject invention include those described in U.S. Pat. No. 6,251,588; the disclosure of which is herein incorporated by reference.

Utility

The above-described methods and devices programmed to practice the same may be used to identify probe nucleic acids to be produced on surfaces of any of a variety of different substrates, including both flexible and rigid substrates, e.g., in the production of nucleic acid arrays. Preferred materials provide physical support for the deposited material and endure the conditions of the deposition process and of any subsequent treatment or handling or processing that may be encountered in the use of the particular array. The array substrate may take any of a variety of configurations ranging from simple to complex. Thus, the substrate could have generally planar form, as for example, a slide or plate configuration, such as a rectangular or square disc. In many embodiments, the substrate will be shaped generally as a rectangular solid, having a length in the range of about 4 mm to 200 mm, usually about 4 mm to 150 mm, more usually about 4 mm to 125 mm; a width in the range of about 4 mm to 200 mm, usually about 4 mm to 120 mm, and more usually about 4 mm to about 80 mm; and a thickness in the range of about 0.01 mm to about 5 mm, usually from about 0.1 mm to about 2 mm and more usually from about 0.2 mm to about 1 mm. However, larger or smaller substrates may be and can be used, particularly when such are cut after fabrication into smaller size substrates carrying a smaller total number of arrays 12. Substrates of other configurations and equivalent areas can be chosen. The configuration of the array may be selected according to manufacturing, handling, and use considerations.

The substrates may be fabricated from any of a variety of materials. In certain embodiments, such as for example where production of binding pair arrays for use in research and related applications is desired, the materials from which the substrate may be fabricated should ideally exhibit a low level of non-specific binding during hybridization events. In many situations, it will also be preferable to employ a material that is transparent to visible and/or UV light. For flexible substrates, materials of interest include: nylon, both modified and unmodified, nitrocellulose, polypropylene, and the like, where a nylon membrane, as well as derivatives thereof, may be particularly useful in this embodiment. For rigid substrates, specific materials of interest include: glass; fuse silica; silicon, plastics (for example polytetraflouroethylene, polypropylene, polystyrene, polycarbonate, and blends thereof, and the like); metals (for example, gold, platinum, and the like).

The substrate surface onto which the polynucleotide compositions or other moieties are deposited may be smooth or substantially planar, or have irregularities, such as depressions or elevations. The surface may be modified with one or more different layers of compounds that serve to modify the properties of the surface in a desirable manner. Such modification layers of interest include: inorganic and organic layers such as metals, metal oxides, polymers, small organic molecules and the like. Polymeric layers of interest include layers of: peptides, proteins, polynucleic acids or mimetics thereof (for example, peptide nucleic acids and the like); polysaccharides, phospholipids, polyurethanes, polyesters, polycarbonates, polyureas, polyamides, polyetheyleneamines, polyarylene sulfides, polysiloxanes, polyimides, polyacetates, and the like, where the polymers may be hetero- or homopolymeric, and may or may not have separate functional moieties attached thereto (for example, conjugated).

Arrays

Also provided by the subject invention are novel nucleic acid arrays of produced using the subject methods, as described above. The subject arrays include at one probe, and typically a plurality of different probes of different sequence (e.g., at least about 10, usually at least about 50, such as at least about 100, 1000, 5000, 10,000 or more) immobilized on, e.g., covalently or non-covalently attached to, different and known locations on the substrate surface. A feature of the subject arrays is that at least one of the probes is a probe having a sequence identified according to the present methods, where in many embodiments at least about 5, 10, 50, 100, 500, 1000, 5000, 10000 or more of the probe sequences are sequences identified by the subject methods. Each distinct nucleic acid sequence of the array is typically present as a composition of multiple copies of the polymer on the substrate surface, e.g. as a spot on the surface of the substrate. The number of distinct nucleic acid sequences, and hence spots or similar structures (i.e., array features), present on the array may vary, but is generally at least 2, usually at least 5 and more usually at least 10, where the number of different spots on the array may be as a high as 50, 100, 500, 1000, 10,000 or higher, depending on the intended use of the array. The spots of distinct nucleic acids present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm², where the density may be as high as 10⁶ or higher, but will generally not exceed about 10⁵ spots/cm². In the subject arrays of nucleic acids, the nucleic acids may be covalently attached to the arrays at any point along the nucleic acid chain, but are generally attached at one of their termini, e.g., the 3′ or 5′ terminus.

A feature of the subject arrays is that they include one or more, usually a plurality of, probes whose sequence as been selected according to the subject protocols. Because the sequences of the probes on the arrays are selected according to the above protocols, the probe sequences are ones that exhibit substantially similar performance under a plurality of different differential gene expression protocols. For example, one or more of the probe sequences on the array will provide performance that varies little, if any, between two or more different differential gene expression assays, i.e., it performs substantially similar under a plurality of different experimental conditions. Where the performance parameter used to determine similarity is hybridization efficiency, the magnitude of any difference observed in hybridization efficiency between any two different differential gene expression analysis protocols of a set of such protocols does not vary by more than about 15-fold, and usually by not more than about 10-fold in certain embodiments. In addition, the subject probes of the arrays identified by the subject methods are ones that provide for high specificity and sensitivity, as described above. In many embodiments, at least about 25 number %, such as at least about 50 number %, 75 number % or more, e.g., 90,95 or 99 or more, up to an including 100 number %, of the probes of the array are probes identified by the subject methods.

Utility of Arrays

The subject arrays find use in a variety applications, where such applications are generally analyte detection applications in which the presence of a particular analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the subject methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g. through use of a signal production system, e.g., an isotopic or fluorescent label present on the analyte, etc. The presence of the analyte in the sample is then deduced from the detection of binding complexes on the substrate surface.

Specific analyte detection applications of interest include hybridization assays in which the nucleic acid arrays of the subject invention are employed. In these assays, a sample of target nucleic acids is first prepared, where preparation may include labeling of the target nucleic acids with a label, e.g., a member of signal producing system. Where the arrays include “all-bases-all-layers” control probes, as described above, a collection of labeled control targets is typically included in the sample, where the collection may be made up of control targets that are all labeled with the same label or two or more sets that are distinguishably labeled with different labels, as described above. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

In certain embodiments, the subject methods include a step of transmitting data from at least one of the detecting and deriving steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

As such, in using an array made by the method of the present invention, the array will typically be exposed to a sample (for example, a fluorescently labeled analyte, e.g., protein containing sample) and the array then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER device available from Agilent Technologies, Palo Alto, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

Kits

Kits for use in analyte detection assays are also provided. The kits at least include the arrays of the invention, as described above. The kits may further include one or more additional components necessary for carrying out an analyte detection assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the array assay devices for carrying out an array based assay. Such kits also typically include instructions for use in practicing array based assays.

Kits for use in connection with the probe design protocols of the subject invention may also be provided. Such kits preferably include at least a computer readable medium including programming as discussed above and instructions. The instructions may include installation or setup directions. The instructions may include directions for use of the invention.

Providing software and instructions as a kit may serve a number of purposes. The combinations may be packaged and purchased as a means of upgrading an existing fabrication device. Alternatively, the combination may be provided in connection with a new device for fabricating arrays, in which the software may be preloaded on the same. In which case, the instructions will serve as a reference manual (or a part thereof and the computer readable medium as a backup copy to the preloaded utility.

The instructions of the above-described kits are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

The following examples are offered by way of illustration and not by way of limitation.

### EXPERIMENTAL

I. Preparation of Nucleic Acid Array Having Human-Specific Content:

The probe validation/probe optimization protocol described in Example 1, below, was performed on sequences representing the 17,838 genes found in the June 2002 release of Incyte's Life Seq™ Foundation “full-length” database (available from Incyte Genomics, Palo Alto, Calif.) and returned 17,803 unique optimized probes: 15,377 (86.4%) of the optimized probes were selected using the empirical probe validation methods described above and 2,426 (13.6%) of the optimized probes were selected using computational criteria. Of the 15,377 empirically optimized probes, only 4,400 (28.6%) showed similar gene expression patterns for 10 of the 10 probes tested and 12,590 (71.4%) had at least one of the candidate probes showing distinct patterns. Disparate log ratio values for probes designed to a single target may be caused by a variety of factors that include non-specific hybridization of additional target(s), probe secondary structure or other factors that limit hybridization efficiency, misannotation of target structure (e.g.: intron/exon boundaries) and labeling biases. Since all candidate probes were selected using “in silico” probe selection methods, these results point to a benefit in the empirical probe optimization strategy of the present invention.

### EXAMPLE 1.

1. Filter probes on signal and form the expression matrix for all targets. Output is an expression matrix, with probes arrayed row by row, and experiments arrayed column by column “probes by experiments”. The output contains only probes that survived the filtering.

2. Compute a histogram of all the similarities (i.e. the mutual similarities of all probes to each target) for use by the cluster affinity threshold and the cluster scoring algorithm.

3. Cluster the candidate probes remaining for each target, using the CAST clustering algorithm, using an affinity threshold determined by a certain percentile in the similarity histogram. The input here is an expression matrix corresponding to one target. The output is two vectors: a vector of cluster memberships and a vector of cluster scores.

3. For targets for which the majority cluster is at least 50% of the candidate probes-identify candidate optimal probes from the majority cluster using the “sum of ranked p-values” as a measure of the quality of the gene expression measures across the experimental set. Select an optimal probe from these candidates as the probe showing the highest processed signal intensity (green or red across the experimental set).

4. For targets for which a majority cluster has not been detected, cluster again with a lower threshold. Repeat.

5. Output data on all targets where a probe was not yet selected. Manually analyze these cases.

All process steps are performed by a VB.NET application connected to a database (in this case MS Jet, but it could equally be any other relational database). Algorithm steps are performed by combinations of VB.NET code and database SQL statements executed by the VB.NET code.

b. Process Output.

The output of the process will have probes, grouped and marked by transcripts, as rows. Columns will be: cluster membership, cluster size, cluster score and threshold used. Optimal probes will be indicated.

### EXAMPLE 2.

a. Selection Process

1. Filter probes on signal. Output is of the form “probes by experiments”. The output contains only probes that survived the filtering.

2. Compute a histogram of all the similarities (i.e. the mutual similarities of all probes to each target).

3. Cluster the candidate probes remaining for each target, using a threshold determined by a certain percentile in the similarity histogram. The input here is an expression matrix corresponding to one target. The output is two vectors: a vector of cluster memberships and a vector of cluster scores.

3. For targets for which the majority cluster is at least 60% of the surviving candidate probes and twice as large as the second lalargest—select an optimal probe from the majority cluster using the average or median log-ratio p-value.

4. For targets for which a majority cluster has not been detected, cluster again with a lower threshold. Repeat.

5. Output data on all targets where a probe was not yet selected. Manually analyze these cases.

All process steps are performed by a VB.NET application connected to a database (in this case MS Jet, but it could equally be any other relational database). Algorithm steps are performed by combinations of VB.NET code and database SQL statements executed by the VB.NET code.

b. Process Output.

The output of the process will have probes, grouped and marked by transcripts, as rows. Columns will be: cluster membership, cluster size (or percent of total), cluster score and threshold used. Optimal probes will be indicated.

It is evident from the above results and discussion that a new and useful method of designing probes for use on nucleic acid microarrays is provided by the subject invention. Benefits of using probes on arrays that are designed according to the present methods include, but are not limited to: (1) users having a high level of confidence in data derived from microarray probes designed according to the present methods; (2) allowing the use of empirically validated 60-mer oligo probes that provide higher sensitivity that short validated probes; and (3) requiring only one array feature to represent each gene on microarray (and no deletion or mismatch controls), thereby provide for more substrate array for more array features for additional different genes. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of identifying a sequence of a nucleic acid that is suitable for use as a substrate surface immobilized probe for a target nucleic acid, said method comprising:
(a) identifying a plurality of candidate probe sequences for said target nucleic acid based on at least one selection criterion;
(b) empirically evaluating each of said candidate probe sequences under a plurality of different experimental sets to obtain a collection of empirical data values for each of said candidate nucleic acid probe sequences for each of said plurality of different experimental sets;
(c) clustering said candidate probe sequences into one or more groups of candidate probe sequences based on each candidate probe sequence's collection of empirical data values, wherein each of said one or more groups exhibits substantially the same performance across said plurality of experimental sets;
(d) selecting one of said one or more groups based on at least one criterion; and
(e) choosing a candidate probe sequence from said selected group to as said sequence of said nucleic acid that is suitable for use as a substrate immobilized probe for said target nucleic acid.

2. The method according to claim 1, wherein said at least one selection criterion employed in said identifying step (a) is chosen from:
(i) proximity to the 3′ end of said target nucleic acid's corresponding mRNA transcript;
(ii) base composition; and
(iii) lack of homology to other expressed sequences of said target nucleic acid's organism.

3. The method according to claim 2, wherein all three of said selection criteria (i), (ii) and (iii) are employed is said identifying step (a).

4. The method according to claim 3, wherein said identifying step (a) is further characterized by employing parameters that minimize the number of identified candidate probe sequences that overlap with each other.

5. The method according to claim 1, wherein said empirically evaluating step (b) comprises for each member of said plurality of different experimental conditions:
(i) providing an array of candidate nucleic acid probes immobilized on a surface of a solid support, wherein said array includes a substrate surface immobilized nucleic acid candidate probe for each of said identified candidate probe sequences; and
(ii) subjecting said array to said member of said plurality of different experimental sets.

6. The method according to claim 5, wherein each member of said plurality of different experimental condition is a different tissue/cell line differential gene expression assay.

7. The method according to claim 1, said clustering step (c) comprises:
(i) obtaining an expression vector for each of said candidate probe sequences using said candidate sequence's collection of empirical data values;
(ii) deriving a similarity matrix for the set of said candidate probe sequences from said candidate probe sequences' expression vectors; and
(iii) grouping said candidate probe sequences based on their derived similarity.

8. The method according to claim 7, wherein those candidate probes that have substantially similar expression patterns are grouped together.

9. The method according to claim 1, wherein the clustering step employs an affinity threshold or another stringency controlling parameter.

10. The method according to claim 1, wherein said at least one criterion employed in said selecting step (d) is chosen from affinity threshold and cluster size.

11. The method according to claim 10, wherein said at least one criterion employed includes both affinity threshold and cluster size.

12. The method according to claim 1, wherein said choosing step (e) comprises choosing a probe sequence from said selected group whose empirical data values meet a minimum performance metric.

13. The method according to claim 12, wherein said minimum performance metric is chosen from signal intensity, confidence measure of the observed expression value, or a combination thereof.

14. The method according to claim 1, wherein at least some of said steps are carried out by a computational analysis system.

15. A computer-readable medium having recorded thereon a program that identifies a sequence of a nucleic acid that is suitable for use as a substrate surface immobilized probe for a target nucleic acid according to the method of claim 1.

16. A computational analysis system comprising a computer-readable medium according to claim 15.

17. A method of producing a nucleic acid array, said method comprising:
producing at least two different probe nucleic acids immobilized on a surface of a solid support, wherein at least one of said at least two different probe nucleic acids has a sequence of nucleotides identified according to the method of claim 1.

18. The method according to claim 17, wherein said at least two different probe nucleic acids are produced on said surface of said solid support by synthesizing said probe nucleic acids on said surface.

19. The method according to claim 17, wherein said at least two different probe nucleic acids are produced on said surface of said solid support by depositing said at least two different probe nucleic acids onto said surface of said solid support.

20. A nucleic acid array produced according to the method of claim 17.

21. A method of detecting the presence of a nucleic acid analyte in a sample, said method comprising:
(a) contacting a nucleic acid array according to claim 20 having a nucleic acid probe that specifically binds to said nucleic acid analyte with a sample suspected of comprising said analyte under conditions sufficient for binding of said analyte to said nucleic acid ligand on said array to occur; and
(b) detecting the presence of binding complexes on the surface of said array to detect the presence of said analyte in said sample.

22. The method according to claim 21, wherein said method further comprises a data transmission step in which a result from a reading of the array is transmitted from a first location to a second location.

23. The method according to claim 22, wherein said second location is a remote location.

24. A method comprising receiving a transmitted result of a reading of an array obtained according to the method claim 20.

25. A kit for identifying a sequence of a nucleic acid that is suitable for use as a substrate surface immobilized probe for a target nucleic acid, said kit comprising:
(a) an algorithm that identifies a sequence of a nucleic acid that is suitable for use as a substrate surface immobilized probe for said target nucleic acid according to the method according to claim 1, wherein said algorithm is present on a computer readable medium; and
(b) instructions for using said algorithm to identify said sequence of a nucleic acid that is suitable for use as a substrate surface immobilized probe for said target nucleic acid.
